(19) United States
(12) Patent Application Publication
(10) Pub. No.: US 2011/0171640 A1
(43) Pub. Date: Jul. 14, 2011

(54) Method for isolating cell free apoptotic or fetal nucleic acids

(75) Inventors: Ram Bhatt, San Diego, CA (US); Wen-Hua Fan, San Diego, CA (US)

(73) Assignee: Novartis Vaccines and Diagnostics, Inc., Emeryville, CA (US)

(21) Appl. No.: 12/865,380

(22) PCT Filed: Feb. 6, 2009
(86) PCT No.: PCT/US09/33375

§ 371 Date: Mar. 21, 2011

Related U.S. Application Data
(60) Provisional application No. 61/028,064, filed on Feb. 12, 2008.

(51) Int. Cl. C12Q 1/68; C07H 21/00; C07K 17/00

(52) U.S. Cl. 435/6.11; 536/25.4; 530/391.1

(57) ABSTRACT

The present invention provides methods for isolating cell free nucleic acid, e.g., apoptotic or fetal nucleic acids and methods of detecting neoplastic cells or identifying the genetic composition of a fetus. The invention also provides magnetic particles comprising an anti-DNA antibody, and kits comprising the magnetic particles.

### RELATED APPLICATIONS

This application is a submission under 35 U.S.C. 371 of PCT/US2009/033375, filed Feb. 6, 2009 which claims priority to U.S. Provisional Application No. 61/028,064, filed Dec. 2, 2008, all of which are hereby incorporated by reference in their entirety.

### BACKGROUND OF THE INVENTION

Prenatal testing or screening is usually performed to determine the gender of the fetus or to detect genetic disorders and/or chromosomal abnormalities in the fetus during pregnancy. As of today, over 4000 genetic disorders, caused by one or more faulty genes, have been recognized. Some examples include Cystic Fibrosis, Huntington's Disease, Beta Thalassaemia, Myotonic Dystrophy, Sickle Cell Anemia, Porphyria, and Fragile-X-Syndrome. Chromosomal abnormality is caused by aberrations in chromosome numbers, duplication or absence of chromosomal material, and by defects in chromosome structure. Examples of chromosomal abnormalities are trisomies, e.g., trisomy 16, a major cause of miscarriage in the first trimester, trisomy 21 (Down syndrome), trisomy 13 (Patau syndrome), trisomy 18 (Edwards syndrome), Klinefelter's syndrome (47, XXY), (47, XYY), and (47, XXX); the absence of chromosomes (monosomy), e.g., Turner syndrome (45, X0); chromosomal translocations, deletions and/or microdeletions, e.g., Robertsonian translocation, Angelman syndrome, DiGeorge syndrome and Wolf-Hirschhorn Syndrome.

Currently available prenatal genetic tests usually involve invasive procedures. For example, chorionic villus sampling (CVS) performed on a pregnant woman around 10-12 weeks into the pregnancy and amniocentesis performed at around 14-16 weeks all contain invasive procedures to obtain the sample for testing chromosomal abnormalities in a fetus. Fetal cells obtained via these sampling procedures are usually tested for chromosomal abnormalities using cytogenetic or fluorescent in situ hybridization (FISH) analyses.

While these procedures can be useful for detecting chromosomal aberrations, they have been shown to be associated with the risk of miscarriage. Therefore amniocentesis or CVS is only offered to women perceived to be at increased risk, including those of advanced maternal age (>35 years), those with abnormal maternal serum screening or those who have had a previous fetal chromosomal abnormality. As a result of these tests the percentage of women over the age of 35 who give birth to babies with chromosomal aberrations such as Down syndrome has drastically reduced. However, lack of appropriate or relatively safe prenatal testing or screening for the majority of pregnant women has resulted in about 80% of Down syndrome babies born to women under 35 years of age.

Thus there is a need for non-invasive screening tests for the general population of pregnant women, especially tests directed to identifying fetal chromosomal aberrations as well as other genetic variations, disorders or diseases. This requires non-invasive techniques of isolating fetal nucleic acid that can be used for prenatal genetic screening.

### SUMMARY OF THE INVENTION

The present invention is based, in part, on the discovery that cell free nucleic acids, e.g., nucleic acids from apoptotic or necrotic cells or fetal nucleic acids can be isolated from a biological sample without lysing, or removing, cells in the sample. Accordingly, the present invention provides methods for isolating cell free nucleic acids, e.g., apoptotic nucleic acids or fetal nucleic acids, and methods of identifying the genetic composition of a fetus. The invention also provides magnetic particles comprising an anti-DNA antibody, and kits comprising the magnetic particles.

In one embodiment of the invention, it provides a method of isolating fetal nucleic acid. The method comprises isolating cell free nucleic acid from a biological sample of a maternal host, that contains a cellular component and cell free nucleic acid, without processing the cellular component prior to the isolation.

In another embodiment of the invention, it provides a method of identifying the genetic composition of a fetus. The method comprises isolating fetal nucleic acid according to a method of the invention, and identifying the genetic composition of the fetus based on the isolated fetal nucleic acid.

In yet another embodiment of the invention, it provides a magnetic particle. The magnetic particle comprises an anti-DNA antibody on its surface.

In still another embodiment of the invention, it provides a kit comprising a magnetic particle of the invention.

### BRIEF DESCRIPTION OF THE FIGURES

FIG. 1 shows exemplary steps in the process of attaching an anti-DNA antibody to streptavidin coated magnetic particles.

FIG. 2 shows exemplary steps in the process of attaching an anti-DNA antibody to streptavidin Dynabeads M-280 via Protein G.

FIG. 3 shows exemplary steps in the process of attaching an anti-DNA antibody to aminated Dynabeads.

### DETAILED DESCRIPTION OF THE INVENTION

The present invention is based, in part, on the discovery that cell free nucleic acids, e.g., apoptotic or fetal nucleic acids can be isolated from a biological sample without lysing, or removing, cells in the sample. According to one aspect of the present invention, it provides a method of isolating apoptotic or fetal nucleic acids. The method comprises isolating cell free nucleic acids from a biological sample, e.g., of a subject or maternal host. The term “cell free nucleic acid” as used herein, refers to, and is used interchangeably with, circulating, or free-floating nucleic acid. In one embodiment, cell free nucleic acid includes nucleic acids existing outside of any intact or partially intact cell. In another embodiment, cell free nucleic acid includes nucleic acids existing outside of any intact or partially intact cell, but within a cellular or cell-like component, e.g., within a membrane structure, a mitochondria-like structure, a lipid membrane vesicle, etc.

In general, the term “maternal host” refers to a female subject carrying, i.e., pregnant with, the fetus. Any suitable biological sample of the maternal host, e.g., one containing fetal nucleic acid, can be used in the methods of the invention. Exemplary biological samples include, but are not limited to, whole blood, plasma, serum, urine, cervical mucus, amniotic fluid, or chorionic villus sample. In one embodiment, the maternal biological sample is whole blood. In another embodiment, the biological sample is a cervical mucus sample or a urine sample stored in an aqueous medium. In yet another embodiment, the biological sample is a sample of a medium, e.g., an aqueous medium, containing nucleic acids leached from one or more cervical mucus or urine samples. The aqueous medium can be any medium, for example an aqueous buffer, suitable for storing a cervical mucus or urine sample.

In general, the biological sample, e.g., maternal biological sample contains a cellular component as well as cell free nucleic acid, and the cell free nucleic acid is isolated without processing the cellular component prior to the isolation. The cellular component may, for example comprise cells or, for example, as in the case of plasma or serum, cellular or matrix factors or proteins.

The term “isolate” as used herein, refers to, and is used interchangeably with, separate, capture, sequester, etc. The isolation of the cell free nucleic acid can be carried out by any means now known, or later discovered, for isolating nucleic acid from a biological sample. In one embodiment, the cell free nucleic acids are isolated by contacting the biological sample with a solid surface containing a ligand for nucleic acids. The ligand can be coated or immobilized on the solid surface either directly, or indirectly, for example, via a linker. Methods for attaching ligands to solid surfaces are well known to those skilled in the art and any method now known, or later developed, can be used. In one embodiment, the solid surface is a magnetic particle, a particle contained in a column, e.g., a resin column, a surface of a microchannel, microwell, plate, filter, membrane, or glass slide.

In another embodiment, the ligand can be coated on the surface of an apparatus, e.g., a microflow apparatus. An exemplary microflow apparatus comprises an inlet means, an outlet means, and a microchannel arrangement extending between the inlet and outlet means. The microchannel arrangement can be any microchannel capable of providing a randomized flow path for the biological sample. For example, the microchannel arrangement can include a plurality of transverse separator posts that are integral with a base surface of the microchannel and project therefrom. The posts are generally arranged in a pattern capable of providing a randomized flow path. Examples of microflow apparatuses are described in U.S. application Ser. Nos. 11/458,668 and 11/331,988, both of which are incorporated herein in their entirety. The surface of the microchannel arrangement of the microflow apparatus can be coated partially or entirely, with the ligand.

Exemplary ligands include, but are not limited to, 4′,6′-diamidino-2-phenylindole (DAPI), an acridine, Distamycin, ethidium bromide, 8-methoxypsoralen, diamino-bistetrahydrofuran, an antisense oligonucleotide, a 2′-deoxyribo- or ribonucleotide, a natural or modified oligonucleotide, PNA, LNA, 2′-methoxy-, phosphorothioates, methylphosphonates, or a combination thereof. In one embodiment, the isolated nucleic acid is DNA, and the ligand is a DNA-binding agent, including, but not limited to, an anti-DNA antibody, e.g., a polyclonal anti-DNA antibody, or a monoclonal anti-DNA antibody; a DNA-binding protein; a DNA-binding nucleic acid; and a DNA-binding organic molecule.

The ligand for nucleic acids may be attached to the solid surface either directly, or via a linker. Any suitable linker, e.g., a hydrophilic or hydrophobic polymer can be used. Examples of linkers include, but are not limited to, IgG, protein-G, Protein A, streptavidin, avidin, and NHS-R-Maleimide, where R is (CH₂CH₂O)ₙ, n=1-100; a polynucleotide, polypeptide, polystyrene, polyethyleneimine, etc. In one embodiment, the linker comprises from about 2 to about 50, 75, 100, 125, 150, 200, or 250 atoms, e.g., C, N, S, P and/or O atoms. In another embodiment, the linker comprises a hydrophilic polymer of varying length, for example, polyethlene glycol, polyvinyl alcohol, etc.

According to the methods of the invention, the cell free nucleic acid is isolated without processing the cellular component prior to the isolation. In one embodiment, the cell free nucleic acid is isolated without lysing, or using a means to lyse, cells in the cellular component prior to the isolation. Such means include any means known for manually lysing, or otherwise killing, destroying, etc., cells so as to release nucleic acid from within the cells into the biological sample. Examples of lysing means include, but are not limited to, treating the biological sample with a lysing agent, a detergent or surfactant; heating the biological sample containing the cells; altering the pH of the biological sample containing the cells, e.g., so that the pH is below pH 4, 5, or 6, or above pH 8, 9 or 10.

In another embodiment, the cell free nucleic acid is isolated without removing, for example, by filtering, centrifuging, etc., the cellular component, i.e., the cells or cellular factors or proteins, prior to the isolation.

In general the methods of the invention can be used to isolate cell free nucleic acid that contains at least 0.00001 percent (1×10⁻⁵%) apoptotic or fetal nucleic acids. In another embodiment, the cell free nucleic acid contains at least 0.00005 percent (5×10⁻⁵%) apoptotic or fetal nucleic acids. In yet another embodiment, the cell free nucleic acid contains at least 0.0001 percent (1×10⁻⁴%) apoptotic or fetal nucleic acids. In still another embodiment, the cell free nucleic acid contains about 0.01%, 0.1%, 1%, 5%, 10%, 20%, 30%, 40%, 50%, 60%, 70%, 80%, or 90% apoptotic or fetal nucleic acid.

The methods of the invention can further comprise the step of isolating nucleic acids with less than about 250, 300, 350, 400, 450, 500, 550, or 600 nucleotides in length from the isolated cell free nucleic acids.

According to another aspect of the present invention, it provides a method of identifying the genetic composition of a fetus. The method comprises isolating fetal nucleic acid according to a method of the invention, and identifying the genetic composition of the fetus based on the isolated fetal nucleic acid.

The genetic composition of the fetus can be indicative of the gender of the fetus, or of a condition or disorder in the fetus. In one embodiment, cell free nucleic acid isolated by a method of the invention can be used directly to determine the gender of the fetus. In another embodiment, fetal nucleic acid is isolated from the cell free nucleic acid and the genetic composition of the fetus is identified based on the isolated fetal nucleic acid. Fetal nucleic acid can be isolated from the cell free nucleic acid by any known means.

In an exemplary embodiment, fetal nucleic acid is isolated from the cell free nucleic acid by size fractionation. Nucleic acids that are less than, for example, about 600, 550, 500, 450, 400, 350, 300 or 250 nucleotides in length are isolated from the cell free nucleic acids. Any known means for size fractionation, e.g., gel electrophoresis (e.g., PAGE), HPLC, TLC, or column-based size fractionation can be used to isolate the fetal nucleic acid.

In one embodiment, the genetic composition of the fetus is identified based on the isolated fetal nucleic acid. The genetic composition could be indicative of a condition or disorder in the fetus. Examples of conditions or disorders include, but are not limited to, Cystic Fibrosis, Sickle-Cell Anemia, Beta-thalassemia, Achondroplasia, Preeclampsia, Phenylketonuria, Tay-Scahs Disease, Adrenal Hyperplasia, Fanconi Anemia, Spinal Muscularatrophy, Duchenne's Muscular Dystrophy, Huntington's Disease, Beta Thalassaemia, Myotonic Dystrophy, Fragile-X Syndrome, Down Syndrome, Edwards Syndrome, Patau Syndrome, Klinefelter's Syndrome, Triple X syndrome, XYY syndrome, Trisomy 8, Trisomy 16, Turner Syndrome, Robertsonian translocation, Angelman syndrome, DiGeorge Syndrome, Wolf-Hirschhorn Syndrome, RhD Syndrome, Tuberous Sclerosis, Ataxia Telangieltasia, and Prader-Willi syndrome.

According to yet another aspect of the present invention, it provides a method of detecting neoplastic cells, neoplastic nucleic acids or genetic markers of neoplastic cells, e.g., tumor cells in a subject. The method comprises isolating cell free nucleic acid according to a method of the invention, and detecting the presence or absence of neoplastic nucleic acid, e.g., genetic markers of neoplastic cells such as tumor cells based on the isolated cell free nucleic acid.

According to yet another aspect of the present invention, it provides a magnetic particle. The magnetic particle comprises an anti-DNA antibody on its surface. The anti-DNA antibody could be a monoclonal antibody or a polyclonal antibody. In one embodiment, the magnetic particle comprises a paramagnetic core. In another embodiment, the magnetic particle comprises a core, e.g., a paramagnetic core, surrounded by a material including, but not limited to, glass, polystyrene, polyethylene, silica, nylon, polyacrylate, polyacrylamide, agarose, ceramic sephadex, and sepharose. The anti-DNA antibody could be attached directly to the magnetic particle, or indirectly, i.e., via a linker.

According to still another aspect of the present invention, it provides a kit comprising a magnetic particle of the invention.

### EXAMPLES

The following examples are intended to illustrate, but not to limit, the invention in any manner, shape, or form, either explicitly or implicitly. While they are typical of those that might be used, other procedures, methodologies, or techniques known to those skilled in the art may alternatively be used.

### Example 1

### Preparation of DNA Binding Beads

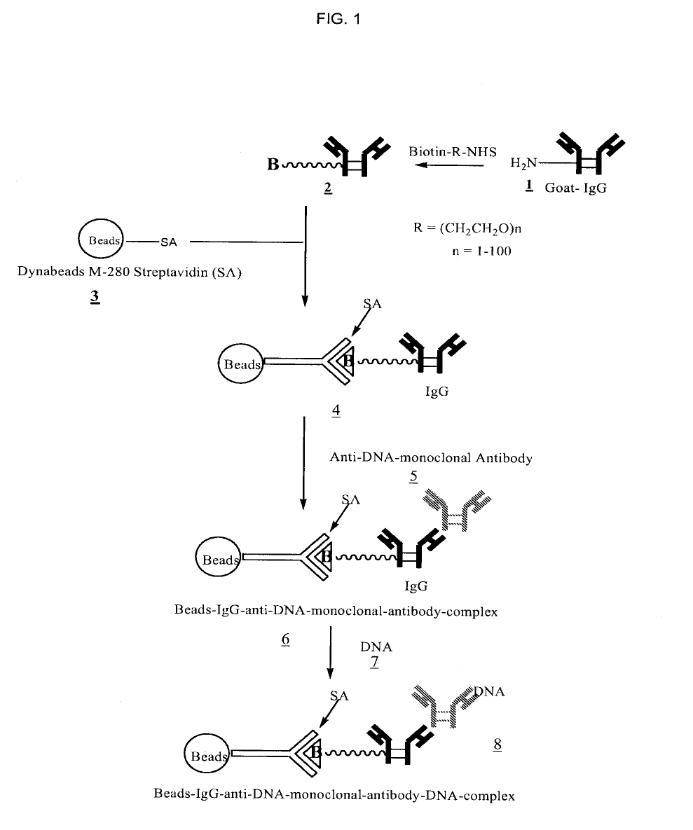

FIGS. 1, 2, and 3 outline the steps involved in preparing magnetic beads conjugated to anti-DNA-antibody through IgG, protein-G and NHS-PEG-Maleimide, respectively.

### Example 2

### Procedure for Isolating Fetal DNA from Maternal Blood

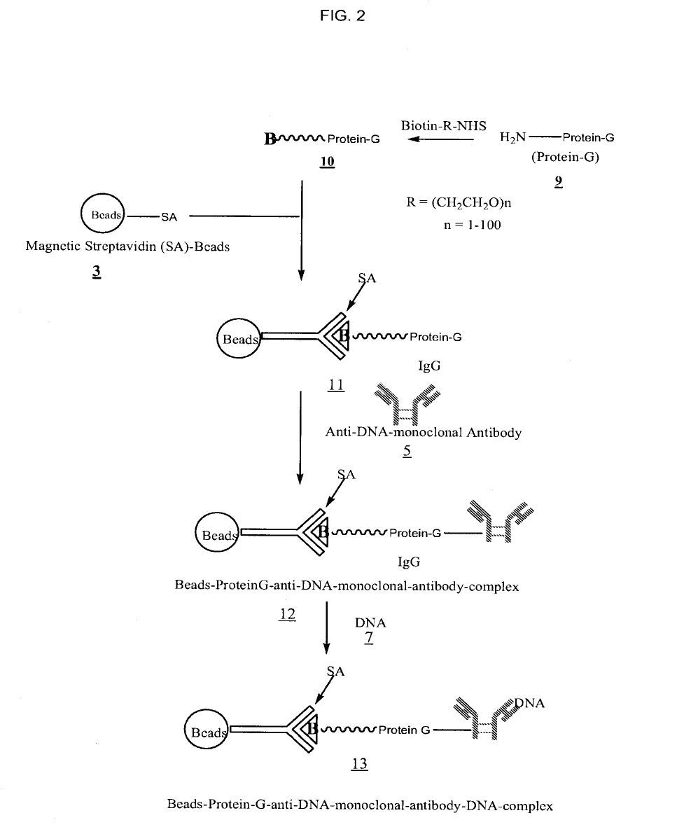

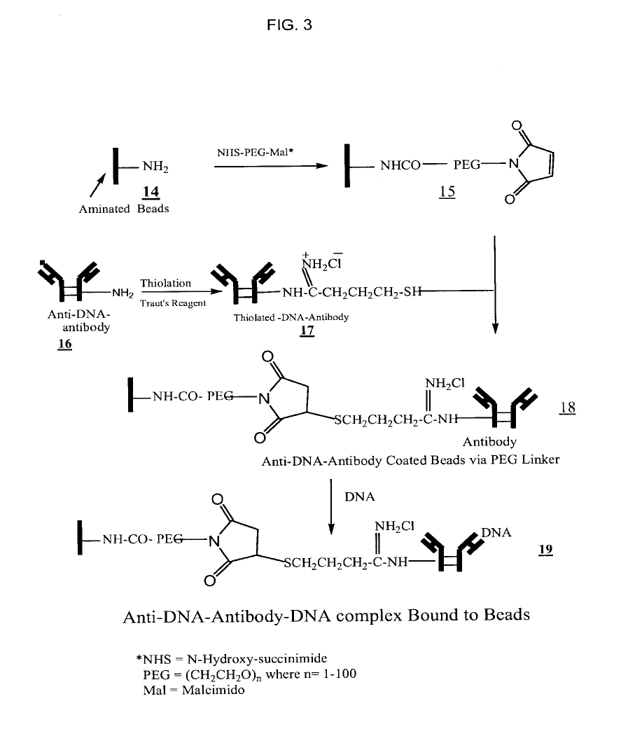

2 ml maternal blood was treated with anti-DNA-antibody coated beads 6 (FIG. 1), 12 (FIG. 2), or 18 (FIG. 3). Enough beads carrying at least 100 μg of anti-DNA-antibody were used. The sample was gently rotated for 15 minutes at room temperature to ensure thorough mixing of the beads with blood. The sample was then placed in a magnetic separator for 1-2 minutes and the supernatant removed. The beads were then washed three times with 2 M NaCl, 10 mM Tris.HCl, 1 mM EDTA (pH 7.0). The beads were then digested with proteinase K in 200 μl of buffer containing 100 mM NaCl, 10 mM Tris.HCl, 25 mM EDTA, 1% SDS (pH 8.0) at 55° C. for 1 hour. After deactivating proteinase K at 95° C. for 10 minutes, the supernatant was ethanol precipitated by adding 2 volumes of absolute ethanol and chilling the sample at −80° C. for 20 minutes. The DNA pellet was rinsed once with 90% ethanol.

### Example 3

### Gender Determination

The DNA from Example 2 was used as a template for determining the gender of the fetus using primers and probes in PCR. After rinsing with 90% ethanol, the DNA pellet was dried, dissolved in 80 μl water and analyzed for fetal gender by PCR. Y-chromosome sequences were detected using one or more TaqMan probes, probes that are dual-labeled, 18-22 base oligonucleotide probes with a reporter fluorophore at the 5′-end and a quencher fluorophore at 3′-end, and one or more primers for Y-chromosome sequence markers.

SRY (Sex-determining Region Y) primers were used to target a sex-determining gene on the Y chromosome, present in humans and other primates. The SRY gene encodes the testis determining factor, which is also referred to as the SRY protein. FCY primers were used to target another common marker in the Y chromosome. The beta-hemoglobin gene, a house-keeping gene that is present in total DNA, was used as an internal control in every PCR reaction. As shown below, all five samples tested have been confirmed by concordant data.

The following controls were used for the PCR reactions:

### Female DNA (Negative Control): 200 ng in 5 μl

### Control Male Genomic DNA (Positive Control):

0 pg control DNA in 5 μl;
7 pg control DNA in 5 μl;
40 pg control DNA in 5 μl
100 pg in μl:
200 pg in μl.

A 96-microwell plate lay-out for 1-11 samples was used for the PCR reaction. The reactions for all controls (male DNA as positive control, female DNA as negative control, and beta-globin), and samples were performed in duplicate for each marker:

Controls with SRY Primers and Probes Samples

| | |
|---|---|
| 1.5 μM SRY Primer mix: 2.5 μl | 1.5 μM SRY Primer mix: 2.5 μl |
| 2.0 μM SRY Probe mix: 2.0 μl | 2.0 μM SRY Probe mix: 2.0 μl |
| Male genomic DNA: 5.0 μl | Extracted Sample DNA: 8.0 μl |
| Water: 3.0 μl | Taqman Universal Mix: 12.5 μl |
| Taqman Universal Mix: 12.5 μl | |

Controls with FCY Primers and Probes Samples

| | |
|---|---|
| 2.0 μM FCY Primer mix: 2.5 μl | 1.5 μM FCY Primer mix: 2.5 μl |
| 3.0 μM FCY Probe mix: 2.5 μl | 2.0 μM FCY Probe mix: 2.0 μl |
| Male genomic DNA: 5.0 μl | Extracted SampleDNA: 8.0 μl |
| Water: 2.5 μl | Taqman Universal Mix: 12.5 μl |
| Taqman Universal Mix: 12.5 μl | |

Controls with β-Globin Primers and Probes Samples

| | |
|---|---|
| 3.0 μM β-Globin Primer mix: 2.5 μl | 3.0 μM β-Globin Primer mix: |
| | 2.5 μl |
| 2.0 μM β-Globin Probe mix: 2.5 μl | 2.0 μM β-Globin Probe mix: 2.5 μl |
| Male genomic DNA: 5.0 μl | Extracted Sample DNA: 7.5 μl |
| Water: 2.5 μl | Taqman Universal Mix: 12.5 μl |
| Taqman Universal Mix: 12.5 μl | |

PCR Running Conditions:

| Step | Temperature | Time | Cycles |
|---|---|---|---|
| Initial Denaturation | 95° C. | 15 min | 1 |
| Denaturation | 94° C. | 30 s | 32 |
| Annealing | 57-61° C. | 60 s | |
| Elongation | 72° C. | 60 s | |
| Final Elongation | 72° C. | 30 min | 1 |

The results of gender testing from whole blood from 2 ml of maternal blood from pregnant women (gestation 7 to 12 weeks) is shown in Table 1.

TABLE 1

| Sample | SRY | FCY | Gender by | Concordant |
|---|---|---|---|---|
| # | (Ct Value) | (Ct Value) | RT-PCR | Data |
| 10584 | 31 | 30.4 | Male | Male |
| 11738 | 37 | 37.5 | Male | Male |
| 11791 | 36.5 | 36 | Male | Male |
| 11915 | 36 | 36 | Male | Male |
| 11917 | 35 | 36 | Male | Male |

Although the invention has been described with reference to the presently preferred embodiment, it should be understood that various changes and modifications, as would be obvious to one skilled in the art, can be made without departing from the spirit of the invention. Accordingly, the invention is limited only by the following claims.

1. A method of isolating fetal nucleic acids comprising:
isolating cell free nucleic acid from a biological sample of a maternal host,
wherein the biological sample contains a cellular component and cell free nucleic acid, and wherein isolation of cell free nucleic acid is carried out without processing the cellular component prior to the isolation.

2. The method of claim 1, wherein the biological sample is whole blood.

3. The method of claim 1, wherein the biological sample is a cervical mucus sample stored in an aqueous medium.

4. The method of claim 1, wherein the biological sample is a urine sample stored in an aqueous medium.

5. The method of claim 1, wherein cell free nucleic acids are isolated by contacting the biological sample with a solid surface containing a DNA-binding agent.

6. The method of claim 5, wherein the solid surface is the surface of a magnetic particle, microchannel, microwell, plate, filter, membrane, or glass slide.

7. The method of claim 5, wherein the DNA-binding agent is an anti-DNA antibody, DNA-binding protein, nucleic acid, or DNA-binding organic molecule.

8. The method of claim 5, wherein the DNA-binding agent is a DNA binding organic molecule selected from the group consisting of acridine, ethidium bromide and DAPI.

9. The method of claim 5, wherein the DNA-binding agent is attached to the solid surface via a linker.

10. The method of claim 9, wherein the linker is selected from the group consisting of IgG, protein-G, Protein A, streptavidin, avidin, polyethlene glycol, polyvinyl alcohol, and NHS-R-Maleimide.

11. The method of claim 9, wherein the linker is a hydrophilic or hydrophobic polymer.

12. The method of claim 10, wherein R is (CH₂CH₂O)ₙ, n=1-100; a polynucleotide; a polypeptide; a polystyrene; or a polyethyleneimine.

13. The method of claim 1, wherein isolation of cell free nucleic acid is carried out without using a means to lyse cells in the cellular component prior to the isolation.

14. The method of claim 1, wherein the isolated cell free nucleic acid is carried out without removing the cellular component prior to the isolation.

15. The method of claim 1, wherein the isolated cell free nucleic acids contains at least 0.0001 percent fetal nucleic acids.

16. The method of claim 1, further comprising isolating nucleic acids with less than 500 nucleotides in length from the isolated cell free nucleic acids.

17. The method of claim 1, further comprising isolating nucleic acids with less than 300 nucleotides in length from the isolated cell free nucleic acids.

18. A method of identifying genetic composition of a fetus comprising:
isolating fetal nucleic acids of a fetus according to the method of claim 1, and
identifying genetic composition of the fetus based on the isolated fetal nucleic acids.

19. The method of claim 18, wherein the genetic composition is indicative of the gender of the fetus.

20. The method of claim 18, wherein the step of isolating fetal nucleic acids further comprises isolating nucleic acids with less than 500 nucleotides in length from the isolated cell free nucleic acids.

21. The method of claim 20, wherein the genetic composition is indicative of a condition or disorder in the fetus.

22. The method of claim 20, wherein the genetic composition is indicative of a disease or disorder selected from the group consisting of Cystic Fibrosis, Sickle-Cell Anemia, Phenylketonuria, Tay-Scahs Disease, Adrenal Hyperplasia, Fanconi Anemia, Spinal Muscularatrophy, Duchenne's Muscular Dystrophy, Huntington's Disease, Myotonic Dystrophy, Beta Thalassaemia, Fragile-X Syndrome, Down Syndrome, Klinefelter's Syndrome, Edwards Syndrome, Patau Syndrome, Triple X syndrome, XYY syndrome, Trisomy 8, Trisomy 16, Turner Syndrome, Robertsonian translocation, Angelman syndrome, DiGeorge Syndrome, Wolf-Hirschhorn Syndrome, RhD Syndrome, Tuberous Sclerosis, Ataxia Telangieltasia, and Prader-Willi syndrome.

23. A magnetic particle comprising an anti-DNA antibody on its surface.

24. The magnetic particle of claim 23, wherein the anti-DNA antibody is a monoclonal antibody.

25. A kit comprising the magnetic particle of claim 23.
